(19) United States
(12) Patent Application Publication
(10) Pub. No.: US 2021/0316017 A1
(43) Pub. Date: Oct. 14, 2021

(54) PHARMACEUTICAL COMBINATION FOR THE TREATMENT OF CANCER

(71) Applicant: Bayer Pharma Aktiengesellschaft, Berlin (DE)

(72) Inventor: Arne SCHOLZ, Berlin (DE)

(73) Assignee: Bayer Pharma Aktiengesellschaft, Berlin (BE)

(21) Appl. No.: 17/103,366

(22) Filed: Nov. 24, 2020

Related U.S. Application Data
(63) Continuation of application No. 15/747,874, filed on Jan. 26, 2018, now abandoned.

(30) Foreign Application Priority Data
Aug. 12, 2015 (EP) 15180755.9

(51) Int. Cl. A61K 51/00; A61K 51/02; A61K 39/395; C07K 16/28; A61P 35/00

(52) U.S. Cl. CPC A61K 51/00; A61K 51/02; A61K 39/3955; A61K 2039/505; A61P 35/00; A61K 39/39541; C07K 16/2818; C07K 16/2827

(57) ABSTRACT

The present invention relates to combinations of at least two components, component A and component B, component A being an immune checkpoint inhibitor, and component B being a pharmaceutically acceptable salt of the alkaline-earth radio-nuclide radium-223, the combination being useful for the treatment or prophylaxis of cancer.

The present invention relates to the use of radium-223 for the enhancement of immunogenicity of tumor cells.

Another aspect of the present invention relates to combinations of at least two components, component A and component B, component A being an immune checkpoint inhibitor, and component B being a pharmaceutically acceptable salt of the alkaline-earth radionuclide radium-223.

Another aspect of the present invention relates to the use of such combinations as described herein for the preparation of a medicament for the treatment or prophylaxis of cancer.

Yet another aspect of the present invention relates to methods of treatment or prophylaxis of a cancer in a subject, comprising administering to said subject a therapeutically effective amount of a combination as described herein.

Further, the present invention relates to a kit comprising a combination of:
one or more components A, as defined herein;
a component B, as defined supra, or a solvate or hydrate thereof; and, optionally
one or more pharmaceutical agents C;
   in which optionally either or both of said components A and 8 are in the form of a pharmaceutical formulation which is ready for use to be administered simultaneously, concurrently, separately or sequentially.

Component A may be administered by the oral, intravenous, topical, local installations, intraperitoneal or nasal route.

Component B preferably is administered by the intravenous route.

### BACKGROUND OF THE INVENTION

Cancer is the second most prevalent cause of death in the United States, causing 450,000 deaths per year. While substantial progress has been made in identifying some of the likely environmental and hereditary causes of cancer, there is a need for additional therapeutic modalities that target cancer and related diseases. In particular there is a need for therapeutic methods for treating diseases associated with dysregulated growth/proliferation.

Cancer is a complex disease arising after a selection process for cells with acquired functional capabilities like enhanced survival/resistance towards apoptosis and a limitless proliferative potential. Thus, it is preferred to develop drugs for cancer therapy addressing distinct features of established tumors.

Prostate cancer is the second most common cancer among men and the second leading cause of cancer death in men in the United States. Most prostate cancers (93%) are found when the disease is confined to the prostate and nearby organs. Overall, most men who develop prostate cancer (99%) are expected to live at least five years after diagnosis. However, for patients diagnosed with metastatic prostate cancer, the five-year survival rate drops to 28%.

Bone is commonly the first area of metastasis for prostate cancer (American Cancer Society: Cancer Facts and Figures 2015). Despite the clinical significance of bone metastatic prostate cancer, only little is known about the molecular mechanisms underlying the development of this disease. Limited therapeutic options are available, and in most cases patients receive only palliative radiation therapy.

Radiation therapy induces hypoxia and the bone is known to be a hypoxic microenvironment. Hypoxia creates an immunosuppressive tumor microenvironment, resulting in recruitment and activation of multiple myeloid and lymphoid immune suppressor cells such as T-regulatory cells, which favors tumor growth and treatment resistance.

The immune system depends on multiple checkpoints or “immunological brakes” to avoid overactivation of the immune system on healthy cells. Tumor cells often take advantage of these checkpoints to escape detection by the immune system. CTLA-4 and PD-1 are checkpoints that have been studied as targets for cancer therapy.

CTLA-4 has been shown to be aberrantly upregulated and present on the surface of T cells in certain cancers, dampening T-cell activation in response to tumor cells. PD-1 is another immunologic checkpoint that has been found to be upregulated in certain tumors; it inhibits T cell function contributing to the tumor's ability to evade the immune system.

Inhibiting a checkpoint on the immune system may enhance the anti-tumor T-cell response.

Currently, different immunotherapeutic approaches are standing their ground as powerful treatment strategies for a wide range of malignant diseases (International Journal of Radiation Biology, April 2015; 91(4): 299-305).

A very prominent and recent example of an outstanding cancer immunotherapy success involves immune checkpoint blockade therapy by monoclonal antibodies (mAb) targeting inhibitory molecules on either immune effector T-cells or tumor cells. Interfering with co-inhibitors has been shown to unleash a powerful antitumor T-cell response (Pardoll: The blockade of immune checkpoints in cancer immunotherapy. Nat Rev Cancer 12: (2012) 252-264).

In 2013, a combination of anti-CTLA4 and anti-PD1 mAb treatment was reported to act synergistically in increasing survival and tumor regression in advanced melanoma patients (Wolchok et al.: Nivolumab plus ipilimumab in advanced melanoma. N Engl J Med 369: (2013) 122-133).

Radiation therapy comes into play as a particularly attractive partner for cancer immunotherapy in empowering the immune system to re-engage in tumor elimination, since recent years have shown a number of mechanisms through which radiotherapy interacts with immunity (Kwilas et al.: Exploiting the untapped potential of immunogenic modulation by radiation in combination with immunotherapy for the treatment of cancer. Front Oncol (2012) 2:104).

Radium-223 chloride is the first in a new class of alpha-particle emitting radiopharmaceuticals for the treatment of patients with castration-resistant prostate cancer with symptomatic bone metastases. Radium-223 is highly targeted to bone due to the chemical similarity between radium and calcium and preferentially incorporates into areas of increased osteoblastic activity. Once deposited, radium-223 releases very cyotoxic, yet short range alpha radiation resulting in the destruction of the metastasis with less damage to the surrounding healthy tissue. It is the short range of the radium-223 activity, 2-10 cells, that leads to its improved safety profile compared to other modes of radiation therapy.

However, from prior art it was unknown whether radium-223 modulates the phenotype of nearby tumor cells as previous radiotherapies have.

### DESCRIPTION OF THE INVENTION

Surprisingly it was observed that a combination of an immune checkpoint inhibitor and a suitable pharmaceutically acceptable salt of the alkaline-earth radionuclide radium-223 act synergistically in tumor regression. The alpha-particle emitting radiopharmaceutical radium-223 is capable of modulating tumor cells that are not directly killed by it in such a way that the tumor cells become more amenable to immune mediated killing.

Therefore, in accordance with a first aspect, the present invention relates to the use of radium-223 for the enhancement of immunogenicity of tumor cells.

In accordance with a another aspect, the present invention relates to the use of radium-223 for preparatory measure of a cancer immunotherapy.

In accordance with a another aspect, the present invention provides combinations of at least two components, component A and component B, component A being an immune checkpoint inhibitor, and component B being a suitable pharmaceutically acceptable salt of the alkaline earth radionuclide radium-223.

The combinations comprising at least two components A and B, as described and defined herein, are also referred to as “combinations of the present invention”.

Further, the present invention relates to:

a kit comprising:
a combination of:
component A: one or more immune checkpoint inhibitors;
component B: a suitable pharmaceutically acceptable salt of the alkaline-earth radionuclide radium-223 or a solvate or a hydrate thereof; and, optionally,
component C: one or more further pharmaceutical agents;
   in which optionally either or both of said components A and B in any of the above-mentioned combinations are in the form of a pharmaceutical formulation/composition which is ready for use to be administered simultaneously, concurrently, separately or sequentially. The components may be administered independently of one another by the oral, intravenous, topical, local installations, intraperitoneal or nasal route.

In accordance with another aspect, the present invention covers the combinations as described supra for the treatment or prophylaxis of cancer.

In accordance with another aspect, the present invention covers the use of such combinations as described supra for the preparation of a medicament for the treatment or prophylaxis of cancer.

The present invention relates to useful forms of the compounds as disclosed herein, such as pharmaceutically acceptable salts. The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, chloride, bromide, iodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, sulfate, tartrate, thiocyanate, tosylate, and undecanoate.

A solvate for the purpose of this invention is a complex of a solvent and a compound of the invention in the solid state. Exemplary solvates would include, but are not limited to, complexes of a compound of the invention with ethanol or methanol. Hydrates are a specific form of solvate wherein the solvent is water.

### Component A of the Combination

Component A is an immune checkpoint inhibitor.

As used herein, the term “immune checkpoint inhibitor” refers to molecules that totally or partially reduce, inhibit, interfere with or modulate one or more checkpoint proteins.

Checkpoint proteins regulate T cell activation or function. Numerous checkpoint proteins are known, such as CTLA-4 and its ligands CD80 and CD86; and PD-1 with its ligands PD-L1 and PD-L2 (Pardoll, Nature Reviews Cancer 12: 252 264, 2012). These proteins are responsible for co-stimulatory or inhibitory interactions of T-cell responses. Immune checkpoint proteins regulate and maintain self-tolerance and the duration and amplitude of physiological immune responses, immune checkpoint inhibitors include antibodies or are derived from antibodies.

As used herein, the term “antibody” includes reference to both glycosylated and nonglycosylated immunoglobulins of any isotype or subclass or to an antigen-binding region thereof that competes with the intact antibody for specific binding, unless otherwise specified, including monoclonal antibodies, bispecific antibodies, minibodies, domain antibodies, synthetic antibodies, antibody mimetics, chimeric antibodies, humanized antibodies, human antibodies, antibody fusions, antibody conjugates, single chain antibodies, antibody derivatives, antibody analogues and fragments thereof, respectively. Also included are immunological fragments of an antibody (e.g., a Fab, a Fab′, a F(ab′)₂, or a scFv), irrespective of whether such antibodies are produced, in whole or in part, via immunization, through recombinant technology, by way of in vitro synthetic means, or otherwise. Thus, the term “antibody” is inclusive of those that are prepared, expressed, created or isolated by recombinant means, such as (a) antibodies isolated from an animal (e.g., a mouse) that is transgenic for human immunoglobulin genes or a hybridoma prepared therefrom, (b) antibodies isolated from a host, cell transfected to express the antibody, e.g., from a transfectoma, (c) antibodies isolated from a recombinant, combinatorial antibody library, and (d) antibodies prepared, expressed, created or isolated by any other means that involve splicing of immunoglobulin gene sequences to other DNA sequences. Such antibodies have variable and constant regions derived from germline immunoglobulin sequences of two distinct species of animals. In certain embodiments, however, such antibodies can be subjected to in vitro mutagenesis (or, when an animal transgenic for human immunoglobulin sequences is used, in vivo somatic mutagenesis) and thus the amino acid sequences of the V_{H} and V_{L} regions of the antibodies are sequences that, while derived from and related to the germline V_{H} and V_{L} sequences of a particular species (e.g., human), may not naturally exist within that species' antibody germline repertoire in vivo. Unless otherwise indicated, the term “antibody” includes, in addition to antibodies comprising two full-length heavy chains and two full-length light chains, derivatives, variants, fragments, and muteins thereof. In some instances “antibody” may include fewer chains such as antibodies naturally occurring in camelids which may comprise only heavy chains.

As used herein, the terms “patient” or “subject” are used interchangeably and mean a mammal, including, but not limited to, a human or non-human mammal, such as a bovine, equine, canine, ovine, or feline. Preferably, the patient is a human.

Accordingly, in one embodiment, the present invention provides a method of treating cancer or initiating, enhancing, or prolonging an anti-tumor response in a subject in need thereof comprising administering to the subject a pharmaceutically acceptable salt of the alkaline-earth radionuclide radium-223 in combination with an agent that is an immune checkpoint inhibitor. In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof.

In a further aspect, the checkpoint inhibitor inhibits a checkpoint protein which may be CTLA-4, PD-11, PD-L2, PDI, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof, in an additional aspect, the checkpoint inhibitor interacts with a ligand of a checkpoint protein which may be CTLA-4, PD-L1, PD-L2, PD-1, B7-H3, B7-H4, BTLA, HVEM, TIM3, GAL9, LAG3, VISTA, KIR, 2B4, CD160, CGEN-15049, CHK 1, CHK 2, A2aR, B-7 family ligands or a combination thereof.

In one embodiment the immune checkpoint inhibitor is an anti-CTLA-4 antibody. In a related embodiment the anti-CTLA-4 antibody is ipilimumab. In another embodiment the anti-CTLA-4 antibody is tremelimumab.

CTLA-4 is an immune checkpoint molecule that down-regulates pathways of T cell activation. CTLA-4 is a negative regulator of T-cell activation. Blockade of CTLA-4 has been shown to augment T-cell activation and proliferation. The combination of radium-223 and the anti-CTLA-4 antibody is intended to enhance T-cell activation. Therefore, the combination of radium-223 and the anti CTLA-4 antibody enhances the destruction of the injected and un-injected/distal tumors, improves overall tumor response, and extends overall survival, in particular where the extension of overall survival is compared to that obtained using an anti-CTLA-4 antibody alone.

In another embodiment, the checkpoint inhibitor is a PD-1 blocker, in another embodiment the checkpoint inhibitor is a PD-L1 blocker. In another embodiment the checkpoint inhibitor is an anti-PD1 antibody. In another embodiment the checkpoint inhibitor is an anti-PD-L1 antibody. In another embodiment the PD-1 blocker is nivolumab. In another embodiment the PD-1 blocker is lambrolizumab. In another embodiment the PD-1 blocker is nivolumab. In another embodiment the PD1 blocker is CT-011. In another embodiment the PD-1 blocker is AMP-224, In another embodiment the PD-L1 blocker is BMS-936559.

PD-1 limits the activity of T cells in peripheral tissues at the time of an inflammatory response to infection and to limit autoimmunity PD-1 blockade in vitro enhances T-cell proliferation and cytokine production in response to a challenge by specific antigen targets or by allogeneic cells in mixed lymphocyte reactions. A strong correlation between PD-1 expression and response was shown with blockade of PD-1 (Pardoll, Nature Reviews Cancer, 32: 252-264, 2032). PD-1 blockade can be accomplished by a variety of mechanisms including antibodies that bind PD-1 or its ligand, PD-L3. Examples of PD-1 and PD-L1 blockers are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In certain embodiments the PD-1 blockers include anti-PD-L1 antibodies. In certain other embodiments the PD-1 blockers include anti-PD-1 antibodies and similar binding proteins such as nivolumab (MDX 1106, BMS 9365S8, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD-1; AMP-2.24 is a fusion protein of B7-DC; an antibody Fc portion; BMS-936559 (MDX-1105-01) for PD-L1 (B7-M1) blockade

There are several PD-1 inhibitors currently being tested in clinical trials. CT-011 is a humanized IgG 1 monoclonal antibody agairrst PD-1. A phase II clinical trial in subjects with diffuse large B-cell lymphoma (DLBCL) who have undergone autologous stem cell transplantation was recently completed. Preliminary results demonstrated that 70% of subjects were progression-free at the end of the follow-up period, compared with 47% in the control group, and 82% of subjects were alive, compared with 62% in the control group. This trial determined that CT-011 not only blocks PD-1 function, but it also augments the activity of natural kill er cells, thus intensifying the antitumor immune response.

BMS 936558 is a fully human IgG4 monoclonal antibody targeting PD 1 agents under in a phase I trial, biweekly administration of BMS-936558 in subjects with advanced, treatment refractory malignancies showed durable partial or complete regressions. The most significant response rate was observed in subjects with melanoma (28%) and renal cell carcinoma (27%), but substantial clinical activity was also observed in subjects with non-small cell lung cancer (NSCLC), and some responses persisted for more than a year. It was also relatively well tolerated; grade ≥3 adverse events occurred in 14% of subjects.

BMS 936559 is a fully human IgG4 monoclonal antibody that targets the PD 1 ligand PD-L1. Phase I results showed that biweekly administration of this drug led to durable responses, especially in subjects with melanoma. Objective response rates ranged from 6% to 17% depending on the cancer type in subjects with advanced-stage NSCLC, melanoma, RCC, or ovarian cancer, with some subjects experiencing responses lasting a year or longer.

MK 3475 is a humanized IgG4 anti-PD-1 monoclonal antibody in phase I development in a five part study evaluating the dosing, safety, and tolerability of the drug in subjects with progressive, locally advanced, or metastatic carcinoma, melanoma, or NSCLC. MPDL 3280A is a monoclonal antibody, which also targets PD-L1, undergoing phase I testing in combination with the BRAF inhibitor vemurafenib in subjects with BRAF V600 mutant metastatic melanoma and in combination with bevacizumab, which targets vascular endothelial growth factor receptor (VEGFR), with or without chemotherapy in subjects with advanced solid tumors.

AMP 224 is a fusion protein of the extracellular domain of the second PD-1 ligand, PD-L2, and IgG1, which has the potential to block the PD-L2/PD-1 interaction. AMP-224 is currently undergoing phase I testing as monotherapy in subjects with advanced cancer. Medi 4736 is an anti-PD-L1 antibody in phase I clinical testing in subjects with advanced malignant melanoma, renal cell carcinoma, NSCLC, and colorectal cancer.

In another embodiment the checkpoint inhibitor is a LAG-3 inhibitor. In another embodiment the LAG-3 inhibitor is IMP321, a soluble Ig fusion protein (Brignone et al., 2007, J. Immunol. 179:4202 4211).

In another embodiment the checkpoint inhibitor is a B7-H3 inhibitor. In another embodiment, the B7-H3 inhibitor is MGA271 (Loo et al., 2012, Clin. Cancer Res. July 15 (38) 3834). In another embodiment the checkpoint inhibitor is a B7-H4 inhibitor.

In another embodiment the checkpoint inhibitor is a TIM3 (T-cell immunoglobulin domain and mucin domain 3) inhibitor (Fourcade et al., 2010, J. Exp. Med. 207:2175- and Sakuishi et al. 2010, J. Exp. Med. 207:2187-94).

### Component B of the Combination

Component B is a suitable pharmaceutically acceptable salt of the alkaline earth radionuclide radium-223.

A suitable pharmaceutically acceptable salt of radium-223 can be, for example, an acid addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2 (4 hydroxybenzoyl) benzoic, camphoric, cinnamic, cyclopentanepropionic, digluconic, 3 hydroxy 2 naphthoic, nicotinic, pamoic, pectinic, persulfuric, 3 phenylpropionic, picric, pivalic, 2 hydroxyethanesulfonate, itaconic, sulfamic, trifluoromethanesulfonic, dodecylsulfuric, ethansulfonic, benzenesulfonic, para toluenesulfonic, methansulfonic, 2 naphthalenesulfonic, naphthalinedisulfonic, camphorsulfonic acid, citric, tartaric, stearic, lactic, oxalic, malonic, succinic, malic, adipic, alginic, maleic, fumaric, D gluconic, mandelic, ascorbic, glucoheptanoic, glycerophosphoric, aspartic, sulfosalicylic, hemisulfuric, or thiocyanic acid, for example.

A preferred suitable pharmaceutically acceptable salt of radium-223 is the dichloride (Ra²²³Cl₂).

Methods for preparation of a physiologically acceptable solution comprising radium-223 are disclosed e.g. in WO 2000/40275(A2), WO 2011/134671(A1), and WO 2011/134672(A1).

Physiologically acceptable solutions comprising radium-223 show a unique mechanism of action as a targeted radiopharmaceutical. They represent a new generation of alpha emitting therapeutic pharmaceuticals based on the natural bone-seeking nuclide radium-223.

Preferably, an aqueous solution of radium-223 chloride (²²³RaCl2) for intravenous injection, sterile and free from bacterial endotoxins is used.

Preferably, the solution is isotonic, containing a sodium citrate buffered saline to physiological pH.

A preferred dosage regimen for radium-223 chloride injection is 50 kBq per kg body weight given at 4 week intervals, as a course consisting of 6 injections. Single radium-223 doses up to 250 kBq per kg body weight were evaluated in a phase I clinical trial. The observed adverse reactions at this dose were diarrhea and reversible myelosuppression (including one case (1/5) of grade 3 neutropenia).

As an example, the aqueous radium-223 dichloride solution may be supplied in a single-dose 10 ml vial which contains a fill volume of 6 ml. This product has a radioactivity concentration of radium-223 of 1,000 kBq/mL (0.03 mCi/ml), corresponding to 0.53 ng/mL of radium at reference date. The active moiety is the alpha particle emitting nuclide radium 223 (half-life is 11.4 days), present as a divalent cation (223Ra2+). The fraction of energy emitted from radium-223 and its daughters as alpha-particles is 95.3%, the fraction emitted as beta-particles is 3.6%, and the fraction emitted as gamma-radiation is 1.1%. The combined energy from the emitted radiation from complete decay of radium-223 and its daughter nuclides is 28.2 MeV.

Radium-223 is to be administered intravenously by qualified personnel as a slow bolus injection. An intravenous access line should be used for administration of radium 223. The line must be flushed with isotonic saline before and after injection of radium-223.

Radium-223 selectively targets areas of increased bone turnover, as in bone metastases, and concentrates by forming a complex with hydroxyapatite. Alpha emission contributes about 93% of the total radiation absorbed dose. The high linear energy alpha particle radiation induces double-strand DNA breaks, resulting in a potent and localized cytotoxic effect in the target areas containing metastatic cancer cells. The short path length (less than 100 micrometers) of the alpha particles minimizes the effect on adjacent healthy tissue such as the bone marrow.

### Optional Component C of the Combination

In accordance with an embodiment, the present invention relates to a combination of any component A mentioned herein with any component B mentioned herein, optionally with any component C mentioned herein.

Component C, can be one or more pharmaceutical agents such as 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 1143269, BAY 1000394, belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumah, glutoxim, goserelin, histamine dihydrochloride, histrelin, hydroxycarbamide, 1-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, raloxifene, raltitrexed, ranimustine, razoxane, refametinib, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin, as well as tumor vaccines like Provenge/Sipleucel-T & ProstVac (rilimogene galvacirepvec/rilimogene glafolivec) & GVAX and the like or combinations thereof.

Alternatively, said component C can be one or more further pharmaceutical agents selected from gemcitabine, paclitaxel, cisplatin, carboplatin, sodium butyrate, 5-FU, doxirubicin, tamoxifen, etoposide, trastumazab, gefitinib, intron A, rapamycin, 17-AAG, U0126, insulin, an Insulin derivative, a PPAR ligand, a sulfonylurea drug, an α-glucosidase inhibitor, a biguanide, a PTP-1B inhibitor, a DPP-IV inhibitor, a 11-beta-HSD inhibitor, GLP-1, a GLP-1 derivative, GIP, a GIP derivative, PACAP, a PACAP derivative, secretin or a secretin derivative.

Optional anti hyper-proliferative agents which can be added as component C to the combination of components A and B of the present invention include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use as component C with the combination of components A and B of the present invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel (when component B is not itself paclitaxel), pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use as component C with the combination of components A and B of the present invention include but are not limited to other anti-cancer agents such as epothilone and its derivatives, irinotecan, raloxifen and topotecan.

Generally, the use of cytotoxic and/or cytostatic agents as component C in combination with a combination of components A and B of the present invention will serve to:
(1) yield better efficacy in reducing the growth of a tumor and/or metastasis or even eliminate the tumor and/or metastasis as compared to administration of either agent alone,
(2) provide for the administration of lesser amounts of the administered chemo-therapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(8) provide a longer time for tumor progression, and/or
(9) yield efficacy and tolerability results at least as good as those of the agents used alone, compared to known instances where other cancer agent combinations produce antagonistic effects.

In a particular embodiment, the present invention relates to a combination of a component A with a component B, optionally with a component C, as mentioned in the Examples Section herein.

Further, the present invention relates to:

a kit comprising:
a combination of:
component A: one or more immune checkpoint inhibitors;
component B: a suitable pharmaceutically acceptable salt of the alkaline-earth radionuclide radium-223 or a solvate or a hydrate thereof; and, optionally,
component C: one or more further pharmaceutical agents;
   in which optionally either or both of said components A and B in any of the above-mentioned combinations are in the form of a pharmaceutical formulation which is ready for use to be administered simultaneously, concurrently, separately or sequentially.

The term “component C” being at least one pharmaceutical agent includes the effective compound itself as well as its pharmaceutically acceptable salts, solvates, hydrates or stereoisomers as well as any composition or pharmaceutical formulation comprising such effective compound or its pharmaceutically acceptable salts, solvates, hydrates or stereoisomers. A list of such readily available agents is being provided further below.

### Commercial Utility

The combinations of the present invention can be used for the treatment or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, particularly in which the uncontrolled cell growth, proliferation and/or survival. Inappropriate cellular immune responses, or inappropriate cellular inflammatory responses, such as, for example, haematological tumours and/or metastases thereof, solid tumours, and/or metastases thereof, e.g. leukaemias, multiple myeloma thereof and myelodysplastic syndrome, malignant lymphomas, breast tumours including and bone metastases thereof, tumours of the thorax including non-small cell and small cell lung tumours and bone metastases thereof, gastrointestinal tumours, endocrine tumours, mammary and other gynaecological tumours and bone metastases thereof, urological tumours including renal, bladder and prostate tumours, skin tumours, and sarcomas, and/or metastases thereof.

One embodiment relates to the use of a combination of the present invention for the preparation of a medicament for the treatment or prophylaxis of a cancer, particularly breast cancer, prostate cancer, multiple myeloma, hepatocyte carcinoma, lung cancer, in particular non-small cell lung carcinoma, colorectal cancer, melanoma, pancreatic cancer and/or metastases thereof.

In one embodiment the invention relates to combinations comprising component A or a pharmaceutically acceptable salt thereof and component B being a pharmaceutically acceptable salt of the alkaline earth radionuclide radium 223 for use in the treatment of cancer indications particularly for such cancer type which is known to form metastases in bone.

Such cancer types are e.g. breast, prostate, lung, multiple myeloma, kidney or thyroid cancer.

Another embodiment relates to the use of a combination according to the present invention for the preparation of a medicament for the treatment or prophylaxis of breast cancer, prostate cancer, multiple myeloma, non-small cell lung cancer and/or metastases thereof, especially wherein the metastases are bone metastases.

In one embodiment the invention relates to a method of treatment or prophylaxis of a cancer, particularly breast cancer, prostate cancer, multiple myeloma, hepatocyte carcinoma, lung cancer, in particular non small cell lung carcinoma, colorectal cancer, melanoma, or pancreatic cancer, in a subject, comprising administering to said subject a therapeutically effective amount of a combination of the present invention.

In another embodiment the invention relates to a method of treatment or prophylaxis of a cancer, particularly breast cancer, prostate cancer, multiple myeloma, hepatocyte carcinoma, lung cancer, in particular non-small cell lung carcinoma, colorectal cancer, melanoma, or pancreatic cancer and/or metastases thereof in a subject, comprising administering to said subject a therapeutically effective amount of a combination of the present invention.

Preferred uses of the combinations of the invention are the treatment of multiple myeloma, lung, breast and prostate cancer, especially castration-resistant prostate cancer (CRPC), and bone metastases.

One preferred embodiment is the use of the combinations of the invention for the treatment of prostate cancer, especially castration-resistant prostate cancer (CRPC) and bone metastases.

One preferred embodiment is the use of the combinations of the invention for the treatment of breast cancer and bone metastases.

Combinations of the present invention might be utilized to inhibit, block, reduce, decrease, etc., cell proliferation and/or cell division, and/or produce apoptosis.

This invention includes a method comprising administering to a mammal in need thereof, including a human, an amount of a component A and an amount of component B of this invention, or a pharmaceutically acceptable salt, isomer, polymorph, metabolite, hydrate, solvate or ester thereof; etc. which is effective to treat the disorder.

Hyper-proliferative disorders include but are not limited, e.g., psoriasis, keloids, and other hyperplasias affecting the skin, benign prostate hyperplasia (BPH), as well as malignant neoplasia. Examples of malignant neoplasia treatable with the compounds according to the present invention include solid and hematological tumors. Solid tumors can be exemplified by tumors of the breast, bladder, bone, brain, central and peripheral nervous system, colon, anum, endocrine glands (e.g. thyroid and adrenal cortex), esophagus, endometrium, germ cells, head and neck, kidney, liver, lung, larynx and hypopharynx, mesothelioma, ovary, pancreas, prostate, rectum, renal, small intestine, soft tissue, testis, stomach, skin, ureter, vagina and vulva. Malignant neoplasias include inherited cancers exemplified by Retinoblastoma and Wilms tumor. In addition, malignant neoplasias include primary tumors in said organs and corresponding secondary tumors in distant organs (“tumor metastases”). Hematological tumors can be exemplified by aggressive and indolent forms of leukemia and lymphoma, namely non-Hodgkins disease, chronic and acute myeloid leukemia (CML/AML), acute lymphoblastic leukemia (ALL), Hodgkins disease, multiple myeloma and T-cell lymphoma. Also included are myelodysplastic syndrome, plasma cell neoplasia, paraneoplastic syndromes, and cancers of unknown primary site as well as AIDS related malignancies.

Examples of breast cancer include, but are not limited to invasive ductal carcinoma, invasive lobular carcinoma, ductal carcinoma in situ, and lobular carcinoma in situ, particularly with bone metastases.

Examples of cancers of the respiratory tract include, but are not limited to small-cell and non small cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head and-neck cancers include, but are not limited to laryngeal, hypopharyngeal, nasopharyngeal, oropharyngeal cancer, lip and oral cavity cancer and squamous cell, lymphomas include, but are not limited to AIDS related lymphoma, non Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Combinations of the present invention might also be used for treating disorders and diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity [Aiello et al. New Engl. J. Med. 1994, 331, 1480; Peer et al. Lab. Invest. 1995, 72, 638], age-related macular degeneration [AMD; see, Lopez et al. Invest. Opththalmol. Vis. Sci. 1996, 37, 855], neovascular glaucoma, psoriasis, retrolental fibroplasias, angiofibroma, inflammation, rheumatoid arthritis (RA), restenosis, in-stem restenosis, vascular graft restenosis, etc. In addition, the increased blood supply associated with cancerous and neoplastic tissue, encourages growth, leading to rapid tumor enlargement and metastasis. Moreover, the growth of new blood and lymph vessels in a tumor provides an escape route for renegade cells, encouraging metastasis and the consequence spread of the cancer. Thus, combinations of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death or apoptosis of such cell types.

In a further aspect, the checkpoint inhibitor and radium-223 are administered simultaneously or sequentially, in either order. In an additional aspect, radium-223 is administered prior to the checkpoint inhibitor.

In one aspect, treatment is determined by a clinical outcome; an increase, enhancement or prolongation of anti-tumor activity by T cells; an increase in the number of anti-tumor T cells or activated T cells as compared with the number prior to treatment or a combination thereof. In another aspect, clinical outcome Is tumor regression; tumor shrinkage; tumor necrosis; anti-tumor response by the immune system; tumor expansion, recurrence or spread or a combination thereof. In an additional aspect, the treatment effect is predicted by presence of T cells, presence of a gene signature indicating T cell inflammation or a combination thereof.

As used herein, the term “treating cancer” is not intended to be an absolute term. In some aspects, the compositions and methods of the invention seek to reduce the size of a tumor or number of cancer cells, cause a cancer to go into remission, or prevent growth in size or cell number of cancer cells. In some circumstances, treatment with the leads to an improved prognosis.

### EXPERIMENTAL SECTION

### Effects of Radium-223 Dichloride Alone and in Combination with Anti-PD-1 mAb on Survival in Treatment Setting in a Mouse Model of Multiple Myeloma

The objective of this study is to observe the effects of combination therapies with radium-223 dichloride (ra-223) and anti-PD-1 mAb on the survival in the syngeneic 5TGM1 mouse multiple myeloma model in treatment setting. The following four experimental groups are included:
1. Rat IgG2a (200 μg per mouse), ip, q3d,
2. Anti-PD-1, (RMP1-14, BioXcell; 200 μg per dose), ip, q3d
3. Ra-223 (dose 300 kBq/kg iv, 5 ml/kg single dose)+Anti-PD-1, (RMP1-14, BioXcell; 200 μg per dose), ip, q3d
4. Ra-223 (dose 300 kBq/kg iv, 5 ml/kg single dose)+rat IgG2a (200 μg per mouse), ip, q3d

Female C57BL/KaLwRij mice are 8-9 weeks of age at the beginning of the study. On day 0, 32 animals are given intravenous of 5TGM2 mouse myeloma cells. Allocation to treatment groups (n=8) is performed by randomization procedure based on serum IgG2b levels at day 21. Ra-223 is administered once at day 22 and administration of rat IgG2a or anti-PD-1 begins at day 25. Administration continues until sacrifice. Animals are sacrificed individually when weight loss over 20%, cachexia, paraplegia or breathing difficulties are observed, or at study day 70. Body weights are determined two times a week starting at day 1. Blood samples are collected on days 21, 35 and at sacrifice for analysis of paraprotein (IgG2b). The development of osteolytic lesions are detected by radiography at day 35 and at sacrifice.

### Radium-223 with Anti-PD-L1 Immunotherapy in Metastatic Prostate Carcinoma Models in Mice

Data show an increase of PD-L1 expression in bone metastatic prostate tumors after local radiation that leads to a better response to anti-PD-L1 treatment. Ra-223 treatment upregulates PD-L1 expression and anti-PD-L1 antibody treatment synergize with Ra-223 treatment.

PD-L1 expression after Ra-223 treatment, and blocking PD-L1 combined with Ra-223 as a treatment for bone metastatic prostate cancer is examined in bone metastatic prostate cancer mice models.

The safety and efficacy of Ra-223 combined with anti-PD-L1 antibody versus Ra 223 or anti-PD-1 antibody alone and radiation therapy as controls are tested. A genetically engineered mouse model (Pten/Smad4-null FVB mice. Ding et al., Nature 2011) is used Treatments are initiated in mice with established tumors. The goal here is to evaluate treatment efficacy. At the experimental endpoint tumor volume, metastatic burden (lung and liver) and mouse survival is measured.

To investigate the mechanisms of action of Ra-223/anti-PD-L1 antibody combination, tumor tissue samples collected in a time-matched fashion are used. The outcomes measured in these experiments are:

Tissue studies of tumor changes after treatment. TUNEL, and Ki-67 immunostaining in bone metastases tissue sections are used. Evaluation of functional vascular density (CD31 and lectin perfusion IHC); hypoxic tissue fraction (pimonidazole staining); intratumoral distribution, number and phenotype of CD8+ and CD4+ T cells, F4/80+ tumor-associated macrophages (TAMs) (M1—versus M2-type), Gr-1+ cells (monocytes versus neutrophils), and PD-L1 expression.

1. A method, comprising using radium-223 to enhance immunogenicity of tumor cells.

2. A combination, comprising at least two components, component A and component B, wherein component A is an immune checkpoint inhibitor, and component B is a suitable pharmaceutically acceptable salt of the alkaline-earth radionuclide radium-223.

3. The combination according to claim 2, in which said pharmaceutically acceptable salt of the alkaline-earth radionuclide radium-223 is radium-223 dichloride.

4. The combination according to claim 2, in which component A is a biologic therapeutic or a small molecule.

5. The combination according to claim 2, in which component A is a monoclonal antibody, a humanized antibody, a fully human antibody, a fusion protein, or a combination thereof.

6. The combination according to claim 2, in which component A is an anti-CTLA-4 antibody.

7. The combination according to claim 2, in which component A is a PD-1 blocker.

8. The combination according to claim 2, in which component A is an anti-PD1 antibody.

9. The combination according to claim 2, in which component A is anti-PD-L1 antibody.

10. The combination according to claim 2, in which component A is a LAG-3 inhibitor.

11. The combination according to claim 2, in which component A is a B7-H3 inhibitor.

12. The combination according to claim 2, in which component A is a TIM3 inhibitor.

13. A kit comprising
a combination of:
component A: one or more immune checkpoint inhibitors; and
component B: a suitable pharmaceutically acceptable salt of the alkaline-earth radionuclide radium-223 or a solvate or a hydrate thereof.

14-16. (canceled)

17. A method of treatment or prophylaxis of cancer in a subject in need thereof, comprising administering to said subject a therapeutically effective amount of a combination of claim 2.

18. The method according to claim 17, in which component B is administered prior to component A.

19. A method of initiating, enhancing, or prolonging an anti-tumor response in a subject in need thereof, comprising administering to the subject a pharmaceutically acceptable salt of the alkaline-earth radionuclide radium-223 in combination with an agent that is an immune checkpoint inhibitor.

20. The kit according to claim 13, wherein the combination comprises one or more further pharmaceutical agents.

21. The kit according to claim 13, wherein the combination is in the form of a pharmaceutical formulation or composition that is ready for use.

22. The kit according to claim 13, wherein component A and component B are administered simultaneously, concurrently, separately, or sequentially.

23. The kit according to claim 13, wherein component A and component B are administered via the oral, intravenous, topical, local installations, intraperitoneal, or nasal route.

24. The method according to claim 17, wherein the cancer is breast cancer, prostate cancer, multiple myeloma, hepatocyte carcinoma, lung cancer, non-small cell lung carcinoma, colorectal cancer, melanoma, or pancreatic cancer, or metastases thereof.

25. The method according to claim 17, wherein the cancer is known to form metastases in bone.
